(19) United States
(12) Patent Application Publication
(10) Pub. No.: US 2002/0193281 A1
(43) Pub. Date: Dec. 19, 2002

(54) Use of fatty acid lower alkyl esters as wetting additives

(76) Inventors: Gerd Mansfeld, Eschershausen (DE); Falk Harzke, Bodenwerder (DE); Jorg Eilers, Hehlen (DE); Dirk Muller, Dassel (DE)

Correspondence Address: BAYER CORPORATION, PATENT DEPARTMENT, 100 BAYER ROAD, PITTSBURGH, PA, 15205, US

(21) Appl. No.: 10/100,435

(22) Filed: Mar. 18, 2002

(30) Foreign Application Priority Data
Mar. 23, 2001 (DE) 10114510.1

(51) Int. Cl. A61K 7/46; B01F 17/00

(52) U.S. Cl. 512/1; 516/198

(57) ABSTRACT

Fatty acid lower alkyl esters can be used as effective constituents in compositions comprising fatty acid lower alkyl esters and perfume compositions, fragrances or perfume oils for setting the viscosity of the composition.

### FIELD OF THE INVENTION

The invention relates to the use of fatty acid lower alkyl esters as wetting additives. The present invention also relates to the use of fatty acid lower alkyl esters for setting the viscosity in perfume compositions. Furthermore the invention relates to mixtures comprising fatty acid lower alkyl esters and perfume compositions or fragrances, and to uses thereof.

### BACKGROUND OF THE INVENTION

Perfume compositions or perfume oils consist of a large number of organic compounds. These compounds, also called fragrances or perfume ingredients, have product-specific properties. These product specific properties include, for example, odor, color, density, optical rotation, refractive index, viscosity, polarity, surface tension and flash point.

During the development of new perfume compositions by the perfumer, the physical and chemical product properties of the ingredients normally play a minor role. The perfumer produces his creations by reference to the odiferous properties of the perfume additives. However, there are applications in which the product-specific properties of perfume compositions play a significant role. This is the case, for example, if perfume compositions or fragrances are discharged via liquid evaporator systems based on piezotechnology or by automatic metering devices, mutual contamination of the fragrances or of the perfume compositions at the metering openings of these devices, which are in close contact with one another, is to be prevented.

A comparison of water and perfume compositions serves to illustrate this problem. When drops of water are applied and placed in close contact to one another, for example small drops of water of about 10 to about 20 mg are placed at a distance of about 3 to about 5 mm from one another on a glass plate, the water drops overall have a considerably lower tendency towards running than perfume compositions under identical experimental conditions. This is due to the fact that perfume compositions sometimes exhibit significant shortcomings in their wetting, flow and contamination behavior.

### SUMMARY OF THE INVENTION

It is therefore an object of the present invention to modify fragrances and/or perfume compositions in such a way that mutual contamination by running or flowing of the respective liquid drops is prevented as much as possible. This modification is achieved with odor-neutral or odorless compositions since these compositions must not adulterate the sensory impression of the fragrances or of the perfume compositions.

Surprisingly, it has now been found that the addition of fatty acid lower alkyl esters having a total of 15 to 20 carbon atoms to perfume oils, perfume compositions or fragrances can significantly improve the wetting, flow and contamination behavior of the mixture comprising fatty acid lower alkyl esters and perfume oils, perfume compositions or fragrances.

Therefore, the present invention provides for the use of fatty acid lower alkyl esters for setting the viscosity of mixtures comprising perfume compositions or fragrances and fatty acid lower alkyl esters, wherein the fatty acid lower alkyl esters contain a total of 15 to 20 carbon atoms.

The present invention further provides mixtures with a viscosity of from about 6 to about 25 mPas, comprising perfume compositions or fragrances and fatty acid lower alkyl esters, wherein the fatty acid lower alkyl esters contain a total of 15 to 20 carbon atoms.

The present invention also provides for the use of these mixtures for application to hard surfaces and for piezoelectrically controlled scent discharge.

### DETAILED DESCRIPTION OF THE PREFERRED EMBODIMENTS

In the present case, lower alkyl is to be understood as meaning methyl, ethyl, n-propyl, isopropyl, n-butyl, isobutyl, sec-butyl and tert-butyl. Preferred lower alkyl radicals are ethyl and isopropyl, particularly preferred is isopropyl.

Fatty acid lower alkyl esters according to the present invention are known in the relevant art per se and are commercially available. Preferably the fatty acid lower alkyl esters have a total of 15 to 20 carbon atoms. More preferably the fatty acid lower alkyl esters have a total of 10 to 19 carbon atoms.

Particularly advantageous fatty acid lower alkyl esters are isopropyl palmitate, ethyl palmitate, ethyl myristate, isopropyl myristate and ethyl stearate. A preferred fatty acid lower alkyl ester is isopropyl myristate (1-metheylethyl tetradecanoate).

In addition, it has now been found that optimum wetting, flow and contamination behavior can be set by the viscosity of the mixture comprising fatty acid lower alkyl esters and perfume compositions, perfume oils or fragrances.

Customarily, the viscosity of the perfume compositions in the fragrance industry is typically in the range from about 1 to about 110 mPas. The viscosity of the mixtures of the present invention comprising fatty acid lower alkyl esters and perfume compositions or fragrances is preferably in the range from about 6 to about 25 mPas, particularly preferably in the range from about 9 to about 15 mpas.

The viscosity of the mixture was determined by using a falling-object viscometer otherwise known as the falling-ball method. The measurements were carried out using a Micro Visko 2 instrument from Haake at 24.5° C. using a 3 mm ball.

The proportion of fatty acid lower alkyl esters in mixtures comprising fatty acid lower alkyl esters and perfume compositions or fragrances is preferably in the range from about 1 to about 80% by weight, more preferably in the range from about 10 to about 50% by weight.

In addition, the solvents customarily used in the perfume oil industry, such as, for example, diethyl phthalate (diethyl 1,2-benzenedicarboxylate), dipropylene glycol (1,1′-oxybis-1-propanol), triethyl citrate (triethyl 2-hydroxy-1,2,3-propanetricarboxylate) can be added in the range from about 1 to about 50% by weight.

The mixtures according to the present invention are suitable for use in piezoelectrically controlled scent discharges, and for application to hard surfaces. The main constituents of the hard surfaces may, for example, be glass, ceramic, metal, porcelain, wood, natural or synthetic polymers or similar materials. In preferred embodiments, mixtures according to the present invention are used as cleaners, in particular as household cleaners. Additionally, mixtures of the present invention are, for example, compositions for cleaning window glass, parquet, and laminate or linoleum flooring.

### EXAMPLES

### Example 1

Table 1 shows the modification of the viscosity using fatty acid lower alkyl esters, specifically isopropyl myristate, of mixtures comprising perfume oils. A narrow viscosity range was set, which is particularly suitable for piezoelectrically controlled scent discharge.

TABLE 1
Viscosity modification of mixtures comprising perfume oils using
isopropyl myristate (data in % by weight).

| Viscosity of | Perfume | Isopropyl | Dipropylene | Set |
|---|---|---|---|---|
| perfume oil | oil | Myristate | glycol | viscosity |
| [mPa * s] | [%] | [%] | [%] | [mPa * s] |
| 2.017 | 42 | 10 | 48 | 9.750 |
| 5.094 | 60 | 10 | 30 | 9.872 |
| 5.498 | 50 | 10 | 30 | 9.970 |
| 8.748 | 70 | 10 | 20 | 10.830 |
| 11.780 | 85 | 15 | 0 | 9.815 |
| 13.200 | 80 | 20 | 0 | 9.900 |
| 14.000 | 75 | 25 | 0 | 9.840 |
| 17.200 | 60 | 40 | 0 | 10.080 |
| 19.680 | 61 | 39 | 0 | 9.881 |
| 21.600 | 60 | 40 | 0 | 10.890 |
| 25.850 | 55 | 45 | 0 | 9.760 |
| 27.250 | 51 | 49 | 0 | 9.643 |
| 31.880 | 53 | 47 | 0 | 9.866 |
| 34.760 | 40 | 60 | 0 | 9.980 |
| 39.830 | 40 | 60 | 0 | 9.728 |
| 54.680 | 40 | 20 | 0 | 10.600 |
| 76.260 | 37 | 63 | 0 | 9.750 |
| 106.100 | 28 | 72 | 0 | 9.649 |

The percentages equaled 100% by using other customary odorless solvents or other fatty acid lower alkyl esters.

Although the invention has been described in detail in the foregoing for the purpose of illustration, it is to be understood that such detail is solely for that purpose and that variations can be made therein by those skilled in the art without departing from the spirit and scope of the invention except as may be limited by the claims.

What is claimed is:

1. A method of setting the viscosity of a mixture, which comprises the steps of adding fatty acid lower alkyl esters to perfume compositions, wherein the fatty acid lower alkyl esters have a total of 15 to 20 carbon atoms.

2. The method according to claim 1, wherein the mixture comprises by weight from about 1 to about 80% of the fatty acid lower alkyl esters.

3. The method according to claim 2, wherein the mixture comprises by weight from about 10 to about 50% of the fatty acid lower alkyl esters.

4. The method according to claim 1, wherein the mixture has a viscosity of from about 6 to about 25 mPas.

5. The method according to claim 4, wherein the mixture has a viscosity of from about 9 to about 15 mPas.

6. The method according to claim 1, wherein the fatty acid lower alkyl ester is selected from the group consisting of isopropyl palmitate, ethyl palmitate, ethyl myristate, isopropyl myristate and ethyl stearate.

7. The method according to claim 6, wherein the fatty acid lower alkyl ester is isopropyl myristate.

8. A composition comprising fatty acid lower alkyl esters and perfume compositions wherein the fatty acid lower alkyl ester has a viscosity of from about 6 to about 25 mPas and wherein the fatty acid lower alkyl esters have 15 to 20 carbons.

9. A composition according to claim 8, wherein the viscosity is from about 9 to about 15 mPas.

10. The composition according to claim 8, wherein the composition comprises by weight from about 1 to about 80% of the fatty acid lower alkyl esters.

11. The composition according to claim 10, wherein the composition comprises by weight from about 10 to about 50% of the fatty acid lower alkyl esters.

12. The composition according to claim 8, wherein the fatty acid lower alkyl ester is selected from the group consisting of isopropyl palmitate, ethyl palmitate, ethyl myristate, isopropyl myristate and ethyl stearate.

13. The composition according to claim 12, wherein the fatty acid lower alkyl ester is isopropyl myristate.
